(19) United States
(12) Patent Application Publication
(10) Pub. No.: US 2009/0170107 A1
(43) Pub. Date: Jul. 2, 2009

(54) DETECTION ASSAY FOR MEAT AND BONE MEAL IN FEED

(76) Inventors: Scott Reaney, Warwickshire (GB); Trevor Conrad Martin, Swansea (GB); Jason Paul Sawyer, Surrey (GB); Saira Cawthraw, Surrey (GB)

Correspondence Address: NIXON & VANDERHYE, PC, 901 NORTH GLEBE ROAD, 11TH FLOOR, ARLINGTON, VA, 22203, US

(21) Appl. No.: 12/297,729

(22) PCT Filed: Apr. 19, 2007
(86) PCT No.: PCT/GB07/01413

§ 371 Date: Feb. 3, 2009

(30) Foreign Application Priority Data
Apr. 19, 2006 (GB) 0607712.7

(51) Int. Cl. C12Q 1/68; C12P 19/34; C07H 21/04

(52) U.S. Cl. 435/6; 435/91.2; 536/23.5

(57) ABSTRACT

The invention concerns methods, kits and nucleic acids involved in detecting animal tissues, for example processed animal proteins (PAPs) or meat and bone meal (MBM), especially in feeds. In one method, a nucleic acid such as DNA is extracted from a sample using a process involving incubating the sample in an incubation buffer, autoclaving the incubated sample, and then mixing the sample with a metal-chelating agent. DNA extracted in this way may then be subjected to amplification using PCR or real-PCR, for example using primer and probe sequences as set forth in SEQ ID NOs 1-21.

This invention relates to methods, kits and nucleic acids involved in detecting and differentiating types of animal tissues, for example processed animal proteins (PAPs) or meat and bone meal (MBM), especially in feeds.

Transmissible spongiform encephalopathies (TSEs) are a group of rare, fatal and transmissible neurodegenerative diseases that include kuru and Creutzfelt-Jakob disease (CJD) in humans, scrapie in sheep, transmissible mink encephalopathy (TME), and chronic wasting disease (CWD) in mule deer and elk. The emergence of bovine spongiform encephalopathy (BSE) in cows was first recognised in the United Kingdom (UK) in 1986 and has now been found in at least 26 countries worldwide. It is considered a worldwide problem not only agriculturally but also as a potential threat to human health.

Rendered animal tissue, which is processed at high temperatures and is also known as meat and bone meal (MBM), has been used as a dietary supplement for farm animals, in particular as a protein supplement to provide essential amino acids to lactating and fast-growing animals. It could either be used directly as feed or as an ingredient in animal feed. Additionally, accidental exposure could arise due to cross-contamination at various stages of handling. BSE developed into an epidemic as a consequence of an intensive fanning practice, i.e. the recycling of animal protein in ruminant feed, and probably originated from a novel source early in the 1970s, possibly a cow or other animal that developed disease as a consequence of a gene mutation. The cases of BSE were the consequences of recycling of cattle infected with BSE itself. The BSE agent was spread in MBM.

To avoid MBM entering the ruminant food chain, a ban was introduced in the UK on feeding ruminant material (for example derived from cattle, sheep, goats or deer) to other ruminants (The Bovine Spongiform Encephalopathy Order 1988 [SI 1988/1039]. This ban was later extended in the UK to include all mammalian proteins to all farmed livestock (excluding fish and horses) (The Bovine Spongiform Encephalopathy (Amendment) Order 1996 [SI 1996/962]). A ban prohibiting the use of mammalian-derived protein in ruminant feed was introduced to the European Union (EU) in 1994 and to the United States of America (USA) in 1997. Since 2000, a European ban has been extended to include proteins derived from mammals, birds and fish for all farmed animals to be used in the production of food.

In order to enforce current legislation it is necessary to be able to detect prohibited proteins in animal feed, especially ruminant feed. Microscopic analysis, which essentially involves the morphological identification of bone fragments in feed material, is currently the only officially recognised method in the EU for the detection of constituents of animal origin in animal feed—see EU Commission Directive 2003/126/EC of 23 Dec. 2003 on an “analytic method for the determination of constituents of animal origin for the official control of feedingstuffs” for the approved microscopic analysis test (“MAT”) protocol. Microscopic analysis permits identification of animal constituents and differentiation between fish and terrestrial material and mammalian versus non-mammalian material. However, microscopic analysis requires an experienced analyst, cannot be used on liquid samples, cannot detect soft tissues, and is a time-consuming and costly method requiring the use of organic solvents for sedimentation of bone fragments.

Other methods being developed for testing animal tissues have included enzyme-linked immunosorbent assay (ELISA), near infrared spectroscopy (NIRS), near infrared microscopy (NIRM), polymerase chain reaction (PCR) and real-time PCR (see review in Gizzi et al., 2003, Rev. Sci. Tech. Off. Int. Epiz. 22: 311-331). ELISAs aim to identify species-specific antigens, without the requirement for bones, via an antibody-based detection system. Drawbacks of the ELISA method include interference from ruminant products such as milk, inhibition by gelatine, and cross-reactivity with other proteins such as plant proteins. NIRS and NIRM require the development of large comparative databases and costly equipment. Particularly for NIRS, accuracy has been a problem. NIRS, NIRM and the ELISA method are not usually suitable for material rendered under conditions beyond the optimised range.

PCR techniques in theory allow the identification of taxon and/or species-specific nucleic acid, particularly deoxyribonucleic acid (DNA). An early example was provided in Wang et al. (2000, Mol. Cell. Probes 14: 1-5), which describes a method involving extraction of total DNA from non-rendered beef meat or bovine MBM spiked into laboratory mouse feed samples. An amount of 0.2 g of the samples was incubated in a solution of 20% Chelex 100 (a chelating resin from BioRad, Hercules, Calif., USA, used for removal of contaminating metal ions that can act as PCR inhibitors) in 1% of the detergent Triton X-100, heated, cooled, and centrifuged, and the supernatant used for PCR amplification of a 271 base pair DNA fragment of bovine mitochondrial DNA encoding for the 3′ part of tRNA^{Lys}, the ATPase subunit 8 and the amino-terminal part of the ATPase subunit 6. The method is stated by the authors to be rapid, simple and consistent.

An example of a real-time PCR assay, involving a ruminant-specific mitochondrial cytochrome b gene probe and primers with an amplicon size of 370 base pairs, was reported in Rensen et al. (2005, Foodborne Pathogens Dis. 2: 152-159; see also WO2005/074522). A pre-sample processing technique for extracting DNA from cattle feed was developed to address problems of high levels of PCR inhibitors in cattle feed. The technique required incubating 10 g spiked feed samples in a cell lysis buffer, applying cell lysate to Whatman FTA™ cards, subsequently treating the cards with RNAse and then purifying DNA using a Chelex 100 extraction kit (the “Instagene” kit) from BioRad (Hercules, Calif., USA). The assay was reported to be able to detect 0.05% (w/w) commercially rendered MBM contaminating material spiked into commercially available cattle feed.

Another real-time PCR assay, based on amplification of a 117 base pair mitochondrial 16S rRNA DNA gene fragment, was developed by Chiappini et al. (2005, J. AOAC Int. 88: 1399-1403). Total DNA was extracted from 100 mg rendered MBM samples using a Wizard Magnetic DNA Purification System for Food obtained from Promega (Madison, Wis., USA). Specificity of their assay was tested on genomic DNA from horse, chicken, turkey, swine, sheep, goat, anchovy, swordfish and bovine material, but a real-time PCR signal was obtained only from goat, sheep and bovine samples. Furthermore, the authors concluded that performance of their real-time PCR assay was greatly affected by the rendering conditions used to produce the MBM tested, and the variability of MBM in terms of composition and rendering conditions prevented the production of suitable reference materials and impeded reliable quantitation of MBM content in feed.

Despite some improvements in PCR techniques, an EU “Intercomparison study for the determination of processed animal proteins including meat and bone meal in animal study” (reported in Gizzi et al., 2004, J. AOAC Int. 87: 1334-1441) found that PCR failed in the determination of total processed animal proteins (PAPs, which includes MBM), as well as in the differentiation of PAPs from mammals, ruminants and pigs, as indicated by a high number of false positive and false negative results from PCR and real-time PCR tests performed in 17 EU participating laboratories. All the common PCR techniques were covered by the participants, including classical PCR with agarose gel electrophoresis, PCR-ELISA and real-time PCR. Sample preparation varied between bespoke or kit extractions, optionally including a recommended grinding step, with the amount of sample used varying between 50 mg to 8 g. The global assessment of the PCR methods was “extremely bad” in terms of performance, and it was concluded that major revisions of the known protocols would be required before any PCR-based technique could be employed as a reliable and robust test for PAPs or MBM.

The present invention addresses problems associated with PCR-based testing for animal tissue in PAPs or MBM, and provides for example an alternative method for extracting DNA and a PCR-based identification technique.

According to a first aspect of the present invention, there is provided a method for extracting nucleic acid (such as DNA) from a sample, comprising the steps of:

(i) incubating between about 30 to 250 g, for example about 35 to 45 g, or about 40 g, of the sample in an incubation buffer; (ii) autoclaving the sample incubated in step (i); and
(iii) mixing the sample or a portion of the sample autoclaved in step (ii) with a metal-chelating agent. The metal-chelating agent may, for example, be a metal-chelating ion-exchange resin.

The method may further comprise the step of:

(iv) sedimenting the metal-chelating agent, such as a metal-chelating ion-exchange resin, from the sample or portion in step (iii) above to produce a supernatant comprising extracted nucleic acid.

The extraction method according to the present invention provides nucleic acid of sufficient quality and quantity, for example to act as a template for amplification in reactions such as PCR and/or real-time PCR. The inventors have found that reproducibility and accuracy of such amplifications reactions are very good (see below). Compared with other prior art extraction techniques, the present method is both easy and effective.

Other suitable amounts of sample for use in the method are between about 30 to up to about 200, 175, 150, 125, 100, 75, or 50 g.

In one embodiment, the incubation buffer may be a phosphate buffer, for example a sodium phosphate buffer at a concentration of about 0.05 to 0.2 M or about 0.1 M.

The incubation buffer may be used at about 500 to 1,500% (v/w) of the mass of the sample, for example about 900% (v/w) of the mass of the sample.

The incubation buffer, for example a sodium phosphate buffer, may have a pH of between about 6.5 and 7.5, or about 7.2.

Incubation may be conducted for about 5 to 60 minutes or for about 10 to 30 minutes, for example for about 15 minutes.

The sample may be autoclaved in step (ii) at about 85° C. to 120° C. or 121° C. or at about 90° C. to 100° C., for example at about 95° C. The autoclave step may last for more than 1 minute, for example for about 5 to 60 minutes or for about 10 to 30 minutes, such as for about 15 minutes.

The autoclave step may proceed at or near atmospheric pressure (rather than at high pressure as used for sterilisation). Autoclaving at or near atmospheric pressure will reduce disruption to the nucleic acid such as DNA being extracted.

As used herein, the phrase “near atmospheric pressure” means a pressure of about 50-150% of atmospheric pressure, for example about 80-120% or 95-105% of atmospheric pressure.

In one embodiment, the autoclave step proceeds at atmospheric pressure for 15 minutes at 95° C.

In particular when animal samples are being used, the autoclave step serves to depellet the sample and/or to break down fat (i.e. to solubilise fats and tallow commonly used for binding feed pellets) and liberate nucleic acid.

The metal-chelating agent, such as an ion-exchange resin, may chelate polyvalent metal ions. The resin may be a styrene divinylbenzene copolymer containing paired iminodiacetate ions, for example Chelex 100.

The metal-chelating agent such as a metal-chelating ion-exchange resin may be sedimented in step (iv) of the method by centrifugation, for example at about 10,000 to 12,000×g, or about 11,000×g.

The sample may be from animal feed, for example ruminant feed.

The sample may contain or be tested for the presence of rendered animal material, for example processed animal proteins (PAPs) or meat and bone meal (MBM).

The sample may comprise animal or ruminant mitochondrial DNA, for example mitochondrial DNA encoding a 16S RNA gene.

In one aspect, the sample comprises DNA from one or more of the group consisting of: animal, ruminant, bovine, ovine, porcine, avian and piscine tissue.

The term “animal” as used herein refers to any vertebrate organism. Animals include domesticated animals (such as cattle, sheep, goats, pigs, chicken, turkey, ducks, geese, quail, cats and dogs) as well as undomesticated animals (such as elk, deer, reindeer and giraffes). Animals also include aquatic species such as fish.

According to a further aspect of the invention, the method as described above, where the sample is animal feed, may comprise the steps of:

(i) incubating between about 35 to 45 g of the sample in a 0.1 M sodium phosphate buffer at a pH of about 7.1 to 7.3 for about 10 to 20 minutes, for example about 15 minutes, optionally at ambient temperature;
(ii) autoclaving the sample incubated in step (i) at or near atmospheric pressure at about 90 to 100° C. for about 10 to 30 minutes, for example for about 15 minutes; and
(iii) mixing a portion of the sample autoclaved in step (ii) with a metal-chelating ion-exchange resin, for example Chelex 100.

The above method may comprise the further step of:

(iv) sedimenting the resin from the portion of the sample in step (iii) by centrifugation at about 10,000 to 12,000×g, or at about 11,000×g.

The supernatant or an aliquot thereof from step (iv) of the method of the invention may be used in an amplification reaction such as a PCR, real-time PCR assay or another amplification reaction as mentioned below.

The method according to the present invention may further comprise the step of:

(v) detecting the presence or absence of nucleic acid, for example DNA such as mitochondrial DNA.

The nucleic acid may be from rendered animal material, for example PAPs or MBM.

The nucleic acid may be detected in step (v) using an amplification reaction (for example PCR or real-time PCR).

The term “amplification reaction” as used herein refers to an enzymatic reaction which results in increased copies of a template nucleic acid. Amplification reactions within the scope of the invention include PCR, reverse transcription PCR(RT-PCR), real-time PCR, real-time RT-PCR(RRT-PCR), ligase chain reaction (LCR), strand displacement amplification (SDA), transcription-mediated amplification, nucleic acid sequence-based amplification (NASBA), rolling circle amplification (RCA) and branched DNA signal amplification (bDNA).

In one aspect, a signalling system is used to detect amplified DNA in the sample or portion of the sample remaining after an amplification reaction. Such a signalling system may be based upon a variety of properties, but in particular will produce visible signals, which are fluorescent, chemiluminescent or bioluminescent. The signalling system may be one that can be detected homogenously, without opening the reaction vessel in which amplification is or has taken place.

Such signalling systems may comprise for example a visible signalling reagent such as a DNA binding agent that emits a different and distinguishable visible when bound to double stranded DNA as compared to when it is free in solution. Examples of such dyes are well known and include ethidium bromide, as well as reagents sold under the trade names of SYBR such as SYBRGreen I or SYBRGold, or other dyes such as YOPRO-1. The presence of significant or high quantities of DNA as indicated by the signal from such a reagent could be indicative that the amplification reaction has proceeded.

Alternatively or additionally, the signalling system may include a labelled probe, which binds specifically to the amplified product. Labels are suitably fluorescent labels, which are detectable following irradiation with light of a suitable wavelength, followed by detection of the resultant emissions from the label. A wide range of fluorescent labels are available commercially, such as rhodamine dyes, fluorescein and cyanine dyes. Further examples of fluorophores include, but are not limited to, fluorescein, isothiocyanate, fluorescein amine, eosin, rhodamine, dansyl, umbelliferone, 5-carboxyfluorescein, 6-carboxyfluorecein (FAM), 2′,4′,1,4,-tetrachlorofluorescein (TET), 2′,4′,5′,7′,1,4-hexachlorofluorescein (HEX), 2′7′-dimethoxy-4′5′-dichloro-6-carboxyfluorescein (JOE), rhodamine, 6 carboxyrhodamine (R6G), NNN′,N′-tetramethyl-6-carboxyrhodamine (TAMRA), 6-carboxy-X-rhodamine (ROX), 4-(4′-dimethylaminophenylazo) benzoic acid (DABCYL), 5-(2′-aminoethyl) aminonaphthalene-1-sulfonic acid (EDANS), 4-acetamido-4′-isothiocyanatostilbene-2,2′ disulfonic acid, acridine, acridine isothiocyanate, r-amino-N->3-vinylsulfonyl)phenyl!naphthalimide-3,5, disulfonate (Lucifer Yellow VS), N-(4-anilino-1-naphthyl)maleimide, anthranilamide, Brilliant Yellow, coumarin, 7-amino-4-methylcoumarin, 7-amino-4-trifluoromethylcouluarin (Coumaran 151), cyanosine, 4′,6-diaminidino-2-phenylindole (DAPI), 5′,5″-diaminidino-2-phenylindole (DAPI), 5′,5″-dibromopyrogallol-sulfonephthalein (Bromopyrogallol Red), 7-diethylamino-3-(4′-isothiocyanatophenyl)-4-methylcoumarin diethylenetriamine pentaacetate, 4,4′-diisothiocyanatodihydro-stilbene-2,2′-disulfonic acid, 4,4′-diisothiocyanatostilbene-2,2′-disulfonic acid, 4-dimethylaminophenylazophenyl-4′-isothiocyanate (DABITC), eosin isothiocyanate, erythrosin B, erythrosin isothiocyanate, ethidium, 5-(4,6-dichlorotriazin-2-yl) aminofluorescein (DTAF), QFITC (XRITC), fluorescamine, IR144, IR1446, Malachite Green isothiocyanate, 4-methylumbelliferone, ortho cresolphthalein, nitrotyrosine, pararosaniline, Phenol Red, B-phycoerythrin, o-phthaldialdehyde, pyrene, pyrene butyrate, succinimidyl 1-pyrene butyrate, Reactive Red 4 (Cibacron® Brilliant Red 3B-A), lissamine rhodamine B sulfonyl chloride, rhodamine B, rhodamine 123, rhodamine X isothiocyanate, sulforhodamine B, sulforhodamine 101, sulfonyl chloride derivative of sulforhodamine 101, (Texas Red), tetramethyl rhodamine, tetramethyl rhodamine isothiocyanate (TRITC), riboflavin, rosolic acid, terbium chelate derivatives, CyS and Cy5.5.

In one aspect, the signalling system comprises a combination of a reporter and quencher label. Examples of donor/quencher pairs which may be used in the present invention are FAM and TAMRA, VIC and TAMRA, FAM and JOE, FAM and ROX, FAM and DABCYL, fluorescein and tetramethylrhodamine, IAEDANS and fluorescein, EDANS and DABCYL, fluorescein and fluorescein, BODIPY FL and BPDIPY FL, FAM and QSY 7 with QSY 9 dyes, respectively. Other suitable combinations of donor/quencher pairs the probe will be understood by the skilled person, or may be determined using routine procedures. The signals generated are read using any convenient detection device, for example an optical system such as a spectrofluorimeter.

As will be understood by the person skilled in the art, amplification reactions include reagents necessary for amplification of the target nucleic acid. Such reagents may include oligonucleotide primers, buffers, a nucleic acid polymerase such as Taq DNA polymerase, and deoxynucleotide triphosphates (dNTPs).

For general PCR and real-time PCR techniques, see for example “PCR Primer: A Laboratory Manual” (2003, Dieffenbach & Dveksler, Eds, Cold Spring Harbor Laboratory Press, Cold Spring Harbor, USA), which is hereby incorporated by reference.

According to another aspect of the invention there is provided a method for amplifying DNA in a sample, comprising the steps of:

(i) extracting DNA from the sample according to the method of the invention defined herein; and
(ii) amplifying the extracted DNA using an amplification reaction such as PCR or real-time PCR.

The DNA amplified in step (ii) above may be from rendered animal material, for example PAPs or MBM.

The PCR or real-time PCR may employ a pair of primers, and in the case of real-time PCR additionally employ a probe, in which the primers and probe are specific for one or more of the following: animal, ruminant, bovine, ovine, porcine, avian and piscine tissue.

The pair of primers and probe may be specific for mitochondrial DNA, for example mitochondrial DNA encoding a 16S RNA gene, from one or more of the group consisting of: animal, ruminant, bovine, ovine, porcine, avian and piscine tissue.

The pair of primers and probe, respectively, may be selected from the group of sequences consisting of SEQ ID NOs 1, 2 and 3 (animal-specific), SEQ ID NOs 4, 5 and 6 (bovine-specific), SEQ ID NOs 7, 8 and 9 (ovine-specific), SEQ ID NOs 10, 11 and 12 (porcine-specific), SEQ ID NOs 13, 14 and 15 (avian-specific), and SEQ ID NOs 16, 17 and 18 (piscine-specific).

The pair of primers and probe may be specific for mitochondrial DNA encoding a 16S RNA gene from one or more of the group consisting of: porcine, avian and piscine tissue. For example, the pair of primers and probe, respectively, may be selected from the group of sequences consisting of SEQ ID NOs 10, 11 and 12 (porcine-specific), SEQ ID NOs 13, 14 and 15 (avian-specific), and SEQ ID NOs 16, 17 and 18 (piscine-specific).

The PCR or real-time PCR may further employ a set of primers, and in the case of real-time PCR additionally employ a probe, wherein the set of primers and probe are specific to DNA exogenous to the sample, for example the sequences as set forth in SEQ ID NOs 19, 20 and 21.

Real-time PCR according to the present invention may be performed using TaqMan probes. Additional labels for use in real-time PCR and other amplification reactions are discussed below.

The method of the present invention in one aspect excludes the use of magnetic DNA purification, for example the Wizard Magnetic DNA Purification System for Food obtained from Promega (Madison, Wis., USA).

The method of the present invention in one aspect excludes the use of RNAse.

The method of the present invention in one aspect excludes the use of Whatman FTA™ card.

The method of the present invention in one aspect excludes the use of Triton X-100 in the incubation buffer.

According to another aspect of the present invention, there is provided a kit for amplifying DNA from a sample using PCR, comprising a pair of primers selected from the group of sequences consisting of: SEQ ID NOs 1 and 2 (animal-specific), SEQ ID NOs 4 and 5 (bovine-specific), SEQ ID NOs 7 and 8 (ovine-specific), SEQ ID NOs 10 and 11 (porcine-specific), SEQ ID NOs 13 and 14 (avian-specific), and SEQ ID NOs 16 and 17 (piscine-specific), and optionally further comprising a set of primers specific for DNA exogenous to the sample, for example the sequences as set forth in SEQ ID NOs 19 and 20.

Also provided is a kit for amplifying DNA from a sample using real-time PCR, comprising a one pair of primers and a probe, respectively, selected from the group of sequences consisting of: SEQ ID NOs 1, 2 and 3 (animal-specific), SEQ ID NOs 4, 5 and 6 (bovine-specific), SEQ ID NOs 7, 8 and 9 (ovine-specific), SEQ ID NOs 10, 11 and 12 (porcine-specific), SEQ ID NOs 13, 14 and 15 (avian-specific), and SEQ ID NOs 16, 17 and 18 (piscine-specific), and optionally further comprising a set of primers and a probe specific to DNA exogenous to the sample, for example the sequences as set forth in SEQ ID NOs 19, 20 and 21.

The kits according to present invention may be used for amplifying rendered animal material, for example PAPs or MBM, from a feed sample.

In another aspect of the invention there is provided an isolated nucleic acid comprising one or more of the sequences set forth in SEQ ID NOs 1-21, for example the sequences set forth in SEQ ID NOs 10-18. Also encompassed by the invention is an isolated nucleic acid having at least 70%, 75%, 80%, 85%, 90%, 95%, 96%, 97%, 98% or 99% sequence identity to any one or more of the sequences set forth in SEQ ID NOs 1-21.

Sequence identity between nucleotide sequences can be determined by comparing an alignment of the sequences. When an equivalent position in the compared sequences is occupied by the same base, then the molecules are identical at that position. Scoring an alignment as a percentage of identity is a function of the number of identical amino acids or bases at positions shared by the compared sequences.

When comparing sequences, optimal alignments may require gaps to be introduced into one or more of the sequences to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties.

Suitable computer programs for carrying out sequence comparisons are widely available in the commercial and public sector. Examples include the Gap program (Needleman & Wunsch, 1970, J. Mol. Biol. 48: 443-453) and the FASTA program (Altschul et al., 1990, J. Mol. Biol. 215: 403-410). Gap and FASTA are available as part of the Accelrys GCG Package Version 11.1 (Accelrys, Cambridge, UK), formerly known as the GCG Wisconsin Package. The FASTA program can alternatively be accessed publically from the European Bioinformatics Institute (http://www.ebi.ac.uk/fasta) and the University of Virginia (http://fasta.biotech.virginia.edu/fasta_www/cgi). FASTA may be used to search a sequence database with a given sequence or to compare two given sequences (see http://fasta.bioch.virginia.edu/fasta_www/cgi/search_form2.cgi). Typically, default parameters set by the computer programs should be used when comparing sequences. The default parameters may change depending on the type and length of sequences being compared. A sequence comparison using the FASTA program may use default parameters of Ktup=2, Scoring matrix=Blosum50, gap=−10 and ext=−2.

In the methods, kits and isolated nucleic acids according to the invention, probes when present may be labelled with any suitable detection means. Thus, for example, the probes comprising the sequences of SEQ ID NOs 3, 6, 9, 12, 15 and 18 need not necessarily be labelled with FAM and TAMRA, and the probe comprising the sequence of SEQ ID NO: 21 need not necessarily be labelled with VIC and TAMRA, but each could be labelled with other means as elaborated above.

The invention also encompasses extracted nucleic acid obtainable according to the method herein described. Such extracted nucleic acid will have novel properties derived from the extraction method.

The present invention will now be described in more detail by way of the following non-limiting examples.

### EXAMPLE 1

In this example, a screening assay was developed, evaluated and validated using animal-specific primers and probes to assist with identification of contaminating rendered animal material in animal feed. The gene used was a 16s rRNA gene present in the mitochondrial genome.

### Materials and Methods

### Real-Time PCR:

The real-time PCR incorporates a fluorogenic probe specific for the identification of a conserved region or species-specific region of the 16s rRNA gene in a mitochondrial genome. During PCR the target is amplified, the probe labelled with a 5′reporter dye (FAM) and a 3′ quencher dye (TAMRA) based on TaqMan technology (Applied Biosystems, Foster City, Calif., USA), binds to the target between the flanking primers. The 5′-3′ nucleolytic activity of the Taq polymerase (AmpliTaq Gold™) hydrolyses the probe releasing the reporter dye from the activity of the quencher dye. The resulting fluorescence is then measured and is directly proportional to the amount of amplicon produced. An increase in the fluorescent signal is only seen if the target sequence is complimentary to the probe and is amplified during PCR. These requirements limit the detection of any non-specific amplification.

### Primers and Probes:

The primers and probes for the target were designed using the following sequences from the GenBank database, avian (X52392), bovine (JO13840), ovine (AF10406), piscine (NC00208) and porcine (AJ002189).

The design of the assays was assisted by the use of Primer Express (Applied Biosystems, Foster City, Calif., USA). For the screening assay, primers and probe were based on a consensus area. Areas of differentiation were chosen for the animal-specific assays, where possible. Table 1 shows the primer and probe sequences for the screening assay and the animal-specific assays, with the expected amplicon size. Forward and Reverse primers were manufactured by Sigma-Genosys Ltd, while the Probe primers were manufactured by Applied Biosystems. The probes were labelled with FAM (5′ end) and TAMRA (3′ end).

TABLE 1

Screening and animal-specific primer and probe
sequences.

| | | | |
|---|---|---|---|
| Primer/ | | Sequence | |
| probe | Name | 5′-3′ | Label |

| | | | | |
|---|---|---|---|---|
| Screening | Forward | MTF1 | AGG GAT AAC AGC | |
| assay | | | GCA ATC | |
| 119 bp | | | (SEQ ID NO: 1) | |
| | Reverse | MTR1 | ATC GTT GAA CAA | |
| | | | ACG AAC C | |
| | | | (SEQ ID NO: 2) | |
| | Probe | MITGEN | TTT ACG ACC TCG | FAM |
| | | | ATG TTG GAT C | |
| | | | (SEQ ID NO: 3) | |
| Avian | Forward | MTFC2 | AGC GCA ATC TCC | |
| (chicken) | | | TCC A | |
| assay | | | (SEQ ID NO: 13) | |
| 110 bp | Reverse | MTR1 | ATC GTT GAA CAA | |
| | | | ACG AAC C | |
| | | | (SEQ ID NO: 14) | |
| | Probe | MITBIRD | ATC AGG ACA ACC | FAM |
| | | | TAA TGG TGC A | |
| | | | (SEQ ID NO: 15) | |
| Bovine | Forward | MTFB | GCG ATT TTA AAG | |
| assay | | | ACT AGA CCC | |
| 108 bp | | | (SEQ ID NO: 4) | |
| | Reverse | MTBRO | TGA ATA GGA TTG | |
| | | | CGC TGT | |
| | | | (SEQ ID NO: 5) | |
| | Probe | MITBOV2 | ATC ACT CTA TCG | FAM |
| | | | CTC ATT GAT CC | |
| | | | (SEQ ID NO: 6) | |
| Ovine | Forward | MTFO | GAG CGA TTT TAA | |
| assay | | | AGA CTA GAC TAA | |
| 110 bp | | | (SEQ ID NO: 7) | |
| | Reverse | MTRBO | TGA ATA GGA TTG | |
| | | | CGC TGT | |
| | | | (SEQ ID NO: 8) | |
| | Probe | MITOV2 | ACC AAA CCA TCG | FAM |
| | | | CTT ATT GAT C | |
| | | | (SEQ ID NO: 9) | |
| Piscine | Forward | MTFH1 | AGC GCA ATC CCC | |
| assay | | | TCT C | |
| 110 bp | | | (SEQ ID NO: 16) | |
| | Reverse | MTR1 | ATC GTT GAA CAA | |
| | | | ACG AAC C | |
| | | | (SEQ ID NO: 17) | |
| | Probe | MITFISH | ATC AGG ACA TCC | FAM |
| | | | TAA TGG TGC A | |
| | | | (SEQ ID NO: 18) | |
| Porcine | Forward | MTFP2 | CAG CGC AAT CCT | |
| assay | | | GTT CTA | |
| 108 bp | | | (SEQ ID NO: 10) | |
| | Reverse | MTRP | GTT GAA CAA ACG | |
| | | | AAC CTT TAA | |
| | | | (SEQ ID NO: 11) | |
| | Probe | MITPOR | ATC AGG ACA CCC | FAM |
| | | | AAA TGG TG | |
| | | | (SEQ ID NO: 12) | |

### Internal Positive Control:

The internal positive control (IPC) involved the amplification of a region of the ampicillin resistance gene commonly found in commercial nucleic acid vectors (pUC18) but not naturally found in animal or plant genomes. It is a non-competitive exogenous control and was included in the validation of the assays to detect false positives that may arise as a result of inhibitory factors present in the sample material or as a result of the sample extraction procedure. The IPC template, primers and probe were added to the TaqMan Master mix to allow multiplex detection. The primers were used at a limiting concentration (0.03 μM) to prevent the IPC product utilising the PCR reagents to the detriment of the 16s rRNA amplification efficiency. In order to detect the amplification of this target in the multiplex assay the probe was labelled at the 5′ end with VIC as the reporter dye and at the 3′ end with TAMRA as the quencher. The primers and probes were designed based on the sequence of pUC18 and are shown in Table 2.

TABLE 2

LPC Primer and probe sequences.

| | | | |
|---|---|---|---|
| Primer/probe | Name | Sequence 5′-3′ | Label |

| | | | |
|---|---|---|---|
| Forward | MITICF1 | GGA TGG CAT GAC AGT | |
| | | AAG AG | |
| | | (SEQ ID NO: 19) | |
| Reverse | MITICR1 | TCG TTG TCA GAA GTA | |
| | | AGT T | |
| | | (SEQ ID NO: 20) | |
| Probe | MITICP | CAG TGC TGC CAT ACC | VIC |
| | | CAT GA | |
| | | (SEQ ID NO: 21) | |

The expected amplicon length is 83 bp.

### PCR and Cycling Conditions Used in the Development and Evaluation:

Real-time PCR was performed and detected on a 7700 Sequence Detector (Applied Biosystems) in a total volume of 25 μl containing 12.5 μl TaqMan Master mix (Applied Biosystems), 0.3 μM of both primers and 0.1 μM of the fluorogenic probe (Applied Biosystems). The cycling conditions were as follows: 50° C. for 2 minutes, followed by 95° C. for 10 minutes, 40 cycles at 95° C. for 15 seconds and 53.6° C. for 1 minute. Each sample was assayed in duplicate.

### PCR and Cycling Conditions Used in the Validation:

Real-time PCR was performed and detected on a 7900HT Sequence Detector (Applied Biosystems) in a total volume of 25 μl containing 12.5 μl TaqMan Master mix (Applied Biosystems), 0.3 μM of both primers and 0.1 μM of the fluorogenic probe for the 16s rRNA assay. For the validation assays, both primers, (0.03 μM) and fluorogenic probe (0.15 μM) (Applied Biosystems) for the IPC were also added to the master mix with the internal positive control template (0.166 ng pUC18). The cycling conditions were the same as those for 7700 Sequence Detector System.

### Samples Used in Development and Evaluation:

### Heat-Treated Tissue

Heat-treated tissue was prepared to generate the effects of sample treatment on the DNA. Thin slices of bovine, ovine, porcine and chicken muscle tissue were homogenised in 0.1 M PBS pH7.2 (10% w/v). This was then heated at temperatures ranging from 90° C. to 133° C. at 3 bar pressure for 20 minutes. DNA was extracted from proteinase K digested and lysed tissue, using a phenol/chloroform method as described in Sambrook et al. (1989, Molecular Cloning: A Laboratory Manual, 2^{nd} edition, Cold Spring Harbor Laboratory Press, Vol. 3, Appendix E: Commonly used techniques in molecular cloning, purification of nucleic acids E3) which is hereby incorporated by reference in its entirety).

### Rendered Material and Quantitative Standards

Bovine, ovine and porcine rendered material was produced from homogenised muscle heated to 128° C. for 20 minutes in simulated rendering autoclaves and then freeze-dried. The fishmeal (based on Micromesistius poutassou) was supplied by United Fish Products (UK) and is commercially available. Crude avian rendered material was prepared from different tissues, since finely ground avian rendered material was not available. The negative plant feed was formulated based on the recommendations of the United Kingdom Agricultural Supply Trade Association (UKASTA) using raw ingredients purchased from BOCM Pauls Ltd. (UK). Table 3 shows the composition of the negative plant feed used to make up the rendered material standards.

TABLE 3
Composition of negative plant feed.

| Constituent | % |
|---|---|
| Wheat | 11.2 |
| Malt Culms | 10.2 |
| Rice bran | 7.9 |
| Wheat feed | 2.9 |
| Citrus pulp | 10.6 |
| Rape extract | 5.9 |
| Palm kernel extract | 11.4 |
| Oat feed | 8.2 |
| Cotton extract | 4.4 |
| Sunflower extract | 5.8 |
| Linseed extract | 4.6 |
| Minerals | 16.9 |

Sample material was produced from 20% rendered material in negative plant feed (50% for avian). Except for the avian feed mixes, these were then ground, using a Retch Ultra centrifugal mill to give a fine particle size of <1 mm. A range (0.5% to 20% bovine, ovine, piscine and porcine, 1% to 50% avian) of quantitative standards were made up to 2 g with negative feed using the 20% and 50% mixes.

### 100 Coded Samples

Coded samples were prepared using avian (chicken), bovine, ovine and porcine rendered material, supplied by Prosper De Mulder (a UK renderer) produced in a rendering facility. The meat and bone meal was pre-sieved using a 2 cm² aperture sieve in order to remove large bone fragments and was aliquotted in the range of 0.001% to 10%. Of these, the 0.1%, 1%, 5% and 10% aliquots were used to make mixed species samples of various combinations using the negative plant feed as above.

### 60 Negative Animal Feed

During the course of assay development, 60 different animal feeds were employed to determine baseline levels of negative controls. There are numerous rendering plants and animal feed producers in the UK, for example. The composition of animal feeds of various suppliers contains a variety of ingredients that are permitted under current regulations. Therefore, it was considered essential to test a variety of these compound feeds in an effort to determine an accurate and sustainable baseline. An ELISA technique (as described in Ansfield et al., 2000, Food Agric. Immunol. 12: 285-297, which is hereby incorporated by reference in its entirety) was used to confirm whether or not the samples were negative for animal material.

### Samples Used in Validation:

A variety of commercially available animal feeds, for example Mendip ewe pellets and Rapid lamb pellets, were used for the validation of the screening and animal-specific assays. All samples were analysed to confirm whether or not they were negative of animal material by ELISA and MAT (microscopic analysis test; see above). In addition, some of these animal feeds were spiked with varying amounts (0.2, 1.0 and 2.0%) of bovine, ovine, porcine, avian (chicken) or fish rendered material, prepared as previously described. Both single species and mixed species samples were produced.

Extraction of DNA from the Rendered Material, Quantitative Standards, 100 Coded Samples and 60 Negative Animal Feed Used During the Development and Evaluation:

The method for extracting DNA from animal feeds was as follows: 1 ml of Chelex solution (20% Chelex-100 [Bio-Rad Laboratories, UK, analytical grade]+1% Nonidet P40) was added to 0.1 g of the feed mix. The samples were mixed vigorously and heated to 95° C. for 15 minutes. They were then placed on ice for 1 minute after which they were spun at 10,000 rpm (about 7,300×g) for 10 minutes. The supernatant was carefully removed to clean tubes and stored at −20° C.

Extraction of DNA from Samples Used in the Validation:

This method was adapted for a large-scale extraction. The increased starting weight allows for a greater representation of the constituents found in the sample feed. 40 g sample was added to 360 ml 0.1 M phosphate buffer and soaked for 15 minutes, after gently shaking, the sample was steam autoclaved at 95° C. for 15 minutes. 1 ml of the supernatant taken from 1 cm below the liquid surface of the sample was added to 0.2 g of Chelex 100 (Bio-Rad Laboratories). The sample was vortexed for 20 seconds and then centrifuged for 10 minutes at 13000 rpm (11,000×g). 400 μl of the supernatant was then transferred to a clean tube and stored at +4° C. until required for testing.

### Results and Discussion

In order to determine the effects of animal feed preparation, DNA extracted from tissue treated at 90° C., 100° C., 110° C., 120° C. and 133° C. at 3 bar pressure for 20 minutes, a simulation of the rendering process, was used in the screening assay. This assay was designed to detect mitochondrial DNA (16s rRNA gene) of avian (chicken), bovine, ovine, piscine and porcine. In the real-time fluorescent PCR, the Ct value is the cycle at which a statistically significant increase in delta Rn (ΔRn) is first detected. The ΔRn is a reliable indicator of the magnitude of the signal generated in the real-time PCR.

In ovine heat-treated tissue using the screening assay, Ct values increased as the temperature of the heat-treatment was increased. For 90° C., the Ct value was 16.80, 100° C. was 17.76, 110° C. was 18.76, 120° C. was 19.03 and for 133° C. the Ct value was 20.39. For the control ovine DNA, the Ct value was 18.70 showing that after 110° C. the DNA became degraded affecting the ability of the PCR to amplify the target. This was consistent with the findings of Ebbehoj and Thomson (1991, Meat Sci. 30: 221-234) who showed that there is little DNA degradation when porcine meat is heated for 30 minutes at 80° C. but significant degradation occurs when the temperature is raised to 120° C. These results show that even though DNA was degraded by the simulated rendering process, the screening assay is a suitable detection method for samples treated up to 133° C. at 3 bars pressure for 20 minutes. This was also seen with DNA extracted from heat-treated avian (chicken), bovine, piscine and porcine material.

In order to evaluate the screening and animal-specific assays, 100 coded samples were used. Samples of known composition, quantitated standards, for each animal species were included in the assay. The results for the screening assay are summarised in Table 4. All samples included at 0.001% were not detected and those at the 0.01% level gave positive results in some instances. Samples included at the 0.1% level were detected in all cases. Based on these results together with those of standards, the assay sensitivity has been determined to be 0.1%. Similarly, when rendered samples were mixed in pairs or in-groups they were detected in each and every case. This coded study showed that the assay could perform the function of a screening assay. In order to fully validate this assay a larger number of samples were screened.

TABLE 4
Summary of results from coded sample study.

| Material | Score |
|---|---|
| Negative Feed (plant matrix) | − |
| Single species 0.001% | − |
| Single species 0.01% | +/− |
| Single species 0.1% | + |
| Single species 1% | + |
| Single species 5% | + |
| Single species 10% | + |
| 10% A with varying % B, P, or O | + |
| 10% B with varying % A, O, or P | + |
| 10% P with varying % A, B or O | + |
| 10% O with varying % A, B, or P | + |
| Totally mixed samples (all species) | + |

Key: (+) All samples positive;
(−) All samples negative;
(+/−) Samples borderline;
B = bovine;
O = ovine;
A = avian;
P = porcine.

All five individual animal-specific assays were unable to detect meat and bonemeal samples included at the 0.001% and 0.01% levels but detected them when included at 0.1%. This coded study established the accuracy of each of the animal-specific assays. There was very good correlation between species present and that detected by each of the animal-specific assays. However, both the bovine and the porcine assays produced one false positive result, i.e. the assays identified a sample containing only ovine and avian material as positive. The detection limits confirmed in this study are consistent with other findings using other PCR methods (Kusama et al., 2004, J. Food Protection 67: 1289-1292; Toyoda et al., 2004, J. Food Protection 67: 2819-2832). Levels of 0.05% have been confirmed using real-time PCR to detect bovine specific material in bovine MBM (Rensen et al., 2005, supra). Our assays show detection limits of 0.1% specific material not only in single mixes but admixtures of samples containing rendered material, a representation of actual field situations of feed contaminates, compared to DNA templates (for example as Lahiff et al., 2002, J. Food Protection 65: 1158-1165; Dalmasso et al., 2004, Mol. Cell Probes 18: 81-87). Again, this coded study showed that the animal specific assays could perform the function of identification. In order to fully validate these assays a larger number of samples were tested.

As there are numerous rendering plants and animal feed producers in the UK, the composition of animal feeds of various suppliers contains a variety of ingredients that are permitted under current regulations. Therefore, it was considered essential to test a variety of these compound feeds in an effort to determine an accurate and sustainable baseline. An ELISA technique was used to designate the samples as negative (see above). Sixty negative animal feeds were tested using the screening assay. The vast majority of the animal feeds (52) were also negative using our screening assay (Ct>35 and/or ΔRn<0.5). However, there were some samples that were positive using the real-time PCR. It is known that the ELISA method works well on samples heated up to 130° C. As the temperature of rendering is increased above this value, the sensitivity of the ELISA declines. Therefore, it is possible that some of these samples were heated to temperatures in excess of 130° C. The other possible explanation for the discrepancy between the two methods is the difference in sensitivity. The ELISA has been determined to detect rendered animal material at the 0.5% inclusion level whereas the present real time assay detects samples at the 0.1% inclusion level.

This discrepancy was investigated further by submitting the samples that were positive by the screening assay together with some samples that were negative, using ELISA, for further analysis using microscopic analysis, the only technique for detecting animal material in animal feed that has been validated for use in the EU. The microscopic analysis results are presented in Table 5.

TABLE 5
Results of microscopic analysis of feed samples showing
differences when analysed by ELISA and TaqMan assays.

| | | TaqMan | Microscopic |
|---|---|---|---|
| Sample No. | ELISA Result | Result | Analysis |
| 2 | − | − | Muscle Fibres |
| 9 | − | + | −* |
| 13 | − | − | − |
| 14 | − | + | Muscle Fibres |
| 15 | − | + | −* |
| 22 | − | − | − |
| 33 | − | + | Muscle Fibres |
| 34 | − | + | Muscle Fibres |
| 36 | − | − | − |
| 42 | − | + | Muscle Fibres |
| 49 | − | + | Muscle Fibres |
| 57 | − | + | Muscle Fibres |

The microscopic analysis results fall into three categories: negative, negative* and muscle fibres. The samples marked negative are true negative. Samples marked negative* are negative for rendered material but contain milk powder, a permitted product. The last category shows the presence of muscle fibres in these samples but the origin of the fibres, i.e. species, is unknown.

Of the 12 samples sent for microscopic analysis, the screening assay showed four samples as negative whereas microscopic analysis showed five samples as negative. With samples 13, 22 and 36, the microscopic analysis and screening methods gave the same result. Sample 2 was negative by the screening assay but muscle fibres were observed in the microscopic analysis. It is possible that in this case the fibres were from a different species to the ones known to be recognized by the screening assay, avian (chicken), bovine ovine, piscine and porcine. Samples 9 and 15 were positive by the screening assay but negative by microscopic analysis, although the presence of milk powder was observed. Milk powder is a permitted additive and has been detected as positive in our screening assay.

The remaining samples 14, 33, 34, 42, 49 and 57 were positive by the screening assay, with the presence of muscle fibres being confirmed by microscopic analysis. In an effort to resolve the question as to the species from which these muscle fibres originated, we subjected these samples to further analysis by utilizing our individual animal-specific assays, i.e. assays that have been designed to detect each of the species under investigation. These results confirmed that 4 of these samples were negative for ovine, bovine, avian (chicken) and porcine but positive for piscine. Therefore, it is highly likely that these muscle fibres originated from piscine. The remaining samples were negative for ovine, porcine avian (chicken) and piscine but positive for bovine material. The screening assay detected milk powder and muscle fibres and the individual animal-specific assays identified the origin of the material. Species-specific identification is important, as the feeding of fishmeal to ruminants has been banned since 2000 in the EU. The existing screening technique (ELISA) failed to detect some of these samples.

In order to fully validate this assay a larger number of samples were tested, using a large-scale DNA extraction procedure. The increased amount of starting material allowed for a greater representation of the constituents found in the samples. The screening and animal-specific assays were validated with the inclusion on an internal positive control shown not to effect the detection of the target gene (16s rRNA) in the screening and animal-specific assays (results not shown). A total of 872 samples were analysed using the screening assay. The negative feed consisted of 286 samples obtained from five different commercial animal feeds. Samples were pre-screened by ELISA and MAT to confirm that they did not contain any banned animal material. Spiking the commercial negative feed with known amounts of specific animal material generated the positive samples.

Of the 286 negative feed samples, 272 were correctly identified as negative and so 14 false positive results were obtained. This corresponds to a 95.1% specificity. False positives identified by the screening assay could potentially be identified with the animal-specific assays. All samples positively spiked with 0.2% and 1% animal material were correctly predicted, giving 100% sensitivity. Of the negative samples spiked with 2% material, one was incorrectly predicted to be negative, giving a specificity of 99.63%. The results are summarised in Table 6.

TABLE 6
Summary of validation screening assay.

| | | Correctly | |
|---|---|---|---|
| Sample | Total | predicted | % Specificity |
| Negative feed | 286 | 272 | 95.1 |
| 0.2% material | 164 | 164 | 100 |
| 1% material | 150 | 150 | 100 |
| 2% material | 272 | 271 | 99.63 |

Of the original 872 samples tested by the screening assay, 273 were identified as negative with a total of 599 samples deemed positive. These 599 positive samples were further analysed using the animal-specific assays. These results are summarised in Table 7.

TABLE 7
Summary of animal-specific assays.

| Species | | | Correctly | % |
|---|---|---|---|---|
| specific | Samples | Total | predicted | Specificity |
| Avian | Negative - no | 433 | 425 | 97 |
| (chicken) | avian | | | |
| | Avian positive | 166 | 161 | 98.1 |
| | samples | | | |
| Bovine | Negative - no | 374 | 371 | 99.2 |
| | bovine | | | |
| | Bovine | 225 | 224 | 99.55 |
| | positive | | | |
| | samples | | | |
| Ovine | Negative - no | 373 | 373 | 100 |
| | ovine | | | |
| | Ovine positive | 226 | 216 | 95.6 |
| | samples | | | |
| Piscine | Negative - no | 422 | 421 | 99.8 |
| | piscine | | | |
| | Piscine | 177 | 171 | 96.6 |
| | positive | | | |
| | samples | | | |
| Porcine | Negative - no | 381 | 378 | 99.2 |
| | porcine | | | |
| | Porcine | 218 | 208 | 95.4 |
| | positive | | | |
| | samples | | | |

Of the 872 samples analysed using the screening assay, 857 were predicted correctly, this gave an overall accuracy of 98.2%. 585 out of 586 spiked samples were identified as being positive. All the samples spiked at a 0.2% level were correctly identified using the animal-specific assays. The 100% specificity at this 0.2% level provides strong evidence that samples containing this level of contaminated material will be identified. The screening assay offers a reliable, more sensitive, robust and cheaper alternative to the existing screening ELISA currently used, and optionally in conjunction with the animal-specific assays could prove to be a valuable method of animal identification.

### EXAMPLE 2

Based on the results of Example 1, we propose the following operating procedure (“OP”) for detection of DNA from rendered animal material in animal feed rations.

### Introduction

### Purpose/Scope of OP

This OP provides a detailed laboratory protocol to test animal feeds for the presence of DNA from rendered animal material using a sensitive real-time PCR assay. The test has 4 key stages: a) Sample preparation for extraction of DNA b) Plate preparation c) PCR reaction d) Data processing and interpretation of results.

### Background Information

European Community wide feed controls (implemented through the TSE (England) Regulations) prohibited the feeding of processed animal proteins to animals which are kept, fattened or bred for the production of food. To eradicate BSE and prevent human form of CJD, a ban on the use of animal proteins in animal feed is in operation. In order to enforce current legislation it is necessary to be able to detect prohibited proteins in animal feed, especially ruminant feed. Microscopic analysis, involving the identification of animal material through analysis of temperature stable solid structures based on morphology, is currently the only official European Union recognised method for the detection of constituents of animal origin in animal feed.

The use of animal DNA as a target in PCR based techniques offers support to current tests as DNA is more resistant to rendering temperatures than proteins. This technique uses real-time PCR to amplify a portion of the 16S rRNA mitochondrial DNA target sequence—any amplicons generated are analysed.

### Principle Involved in Method

DNA is extracted from a 40 gram of feed sample. The feed sample is de-pelleted in a phosphate buffer using a heating step and then a sub-sample is treated with chelex resin. The mixture is vortexed, centrifuged and an aliquot of the supernatant containing DNA is used for testing. Sample DNA is added to Master-mix containing buffer, primers, probes, pUC18 and polymerase enzyme in a test plate. The PCR reaction is carried out in the ABI prism 7900 sequence detector. The system results in a fluorescence if the target is amplified during the PCR reaction. Fluorescence is measured directly within the PCR plate and the results are calculated based on the intensity of the fluorescent signal. Internal control DNA (pUC18) with specific primers and different fluorescent probe is used to assess if there has been inhibition of amplification. Ct (Threshold cycle) values are calculated for each sample during their reaction and compared to a cut off level established per plate based on Ct values of control samples.

### Materials

### Documentation and Software

ABI SDS Software, version 2.0 or later.
TaqMan™ Assay Sheet (see manufacturer's instructions).

### Chemicals and Reagents

Phosphate Buffer pH 7.2+0.1 (0.1M)

Solution A: 14.2 g di-sodium hydrogen orthophosphate (anhydrous), dissolved in 1 litre of distilled water.

Solution B: 15.6 g Sodium dihydrogen orthophosphate (dihydrate), dissolved in 1 litre of distilled water.

Mix 720 ml of solution A with 280 ml of solution B, check pH is 7.2+0.1 (larger volumes can be made for high through put, but should be kept no longer than seven days).
Each new TaqMan reagent should be assessed, as characteristics may vary from batch to batch and between manufacturers. New batches of PCR mastermix components (reagents, probes or primers) should be introduced one at a time and tested in order to determine fitness for purpose. Applied Biosystems' TaqMan Universal PCR Master Mix is currently used. Equivalent reagents from other suppliers that have been shown to be fit for purpose may be used.
pUC18 plasmid DNA (Sigma Catalogue No. D4154)
Molecular biology grade water certified DNA- and RNA-free (Sigma Catalogue No. W4502)
HPLC-purified specific and control primers—see Tables 1 and 2 above.

### Control Samples

Negative Control: Negative Feed Stock comprising of Rape, Soya, Sunflower and Maize in equal proportions. Ingredients supplied by GAFTA.
Positive controls: Bovine, Ovine, Porcine and Avian tissue supplied by Prosper de Mulder. Fish tissue supplied by United Fish Industries.

### Equipment

ABI PRISM 1900HT Sequence Detection instrument and PC.
Electronic balance. Able to measure between 40 g±5 g. (Range 0 to 3 kg)
Electronic balance. Able to measure between 0.2 g±0.02 g. (Range 0 to 100×g)
Centrifuge with fixed rotatable to spin microtubes at 13000 rpm (11,000×g)
Centrifuge for spinning 96 well plates (2000 rpm [700×g])
Range of adjustable single channel pipettes capable of dispensing volumes 0.1-1000 μl accurately.
Multichannel pipette, range 1-10 μl
Laminar flow cabinet or class II safety cabinet
Autoclave, capable of operating at 95° C.±5° C. at atmospheric pressure.

Freezer, <−18° C.

Vortex mixer.

### Consumables

ABI PRISM optical cover compression pads (Applied Biosystems)
ABI PRISM adhesive covers (Applied Biosystems)
Optical 96-well reaction plate (Applied Biosystems)
Filter-barrier tips for pipettors (VWR)
Sterile microfuge tubes 0.6-1.7 ml (Alpha Labs or other reputable suppliers)
Sterile plastic bijoux (VWR)
Autoclavable containers (1 litre volume)
Measuring cylinders of appropriate size.

### Method

### Sample Identification and Handling

Identify each submission by a unique combination of reference number and date of receipt. Sample handling should be in accordance with relevant safety procedures.

### Sample Preparation

To 40 g±5 g of sample add 360 ml of 0.1M phosphate buffer pH 7.2±0.1. Soak for 15 minutes at room temperature in a container suitable for autoclaving. Autoclave at 95±5° C. for 15 minutes. Weigh out 0.2±0.02 g of chelex resin into a 1.7 ml microcentrifuge tube. Remove the samples from autoclave and extract 1 ml of supernatant from approximately 1 cm below the liquid surface. Transfer to the microcentrifuge tube containing chelex.

Vortex the sample at maximum speed for 20 seconds. Centrifuge the samples for 10 minutes±60 seconds at 13,000 rpm (11,000×g). Remove 10 μl of the supernatant and add 90 μl of molecular grade distilled water. Use the extract immediately or hold at <−18° C. prior to testing.

### Preparation of Working Solutions

Primers: Oligonucleotide (Oligo) primers are supplied as lyophilised pellets that need reconstituting and diluting to form stock solutions for aliquots and storage.

Dissolve the oligo pellet in biological grade water to give a 100 μM solution. Mix well by vortexing and allow to stand for 2 minutes at room temperature. Mix again.

Dispense the 100 μM primer stock into 50 μl or lower aliquots in 0.6 ml microfuge tubes. Label the primers and store at <−18° C.

Probes: Resuspend probes in 1 ml biological grade water. Dispense into 50 μl aliquots in 0.6 ml microtubes, label and store at <−18° C.

pUC18: Dilute the pUC18 stock to 1/15000. Dispense into 50 μl aliquots in 0.6 ml microtubes, label and store at <−18° C.

Water: Dispense into 25 ml universals and store them at room temperature.

### Preparing Negative and Positive Controls

Negative Control: 40 g±5 g of negative feed stock processed as set out above and the chelex extract is collected. This negative control is ready to use or may be stored at <−18° C. for 1 year. New batches of negative control material should be tested alongside the old batch in order to establish fitness for purpose.

Positive Control (0.2%): 40 g±5 g of negative feed stock spiked individually with 0.08 g of Bovine/Porcine/Ovine/Avian/Fish meat and bone meal and processed as set out above and the chelex extract is collected. These positive controls are ready to use or may be stored at <−18° C. for 1 year. New batches of positive control material should be tested alongside the old batch in order to establish fitness for purpose.

### Preparing PCR Master-Mix

Depending on the assay, calculate the primer and probe concentrations required and adjust total volumes depending on number of tests (wells) being used. Remove the aliquoted stocks of primers, probe, pUC18 and the TaqMan universal Master-mix from fridge/freezer and allow to reach room temperature. Gently tap the tubes to settle the contents into the bottom of the tubes. Working in the clean room safety cabinet, pipette the required volume of each reagent into a 7 ml bijoux. Do not add the pUC18 at this stage. Return to the test lab and add required volume of pUC18. Once all reagents have been added vortex the master mix for 5 to 10 seconds.

### Setting up TaqMan Plates

For the general screening assay, set out one or more plates to include reactions for several no template controls (NTC's), at least one positive control for each tissue type except for avian tissue which should have at least six reactions, negative controls, and the samples to be tested. For the species specific assay, the same layout as above should be used, except that at least six positive control reactions for the target species are included for calculating Ct cut off values.

Add 23 μl of the master mix (see above) to each well of a 96 well Optical reaction plate. Add 2 μl of the appropriate sample DNA or control DNA to the 96 well plate. Cover the plate with ABI PRISM optical adhesive cover and seal. Centrifuge the plate for 3 s at 2000 rpm (700×g) to settle the contents of the wells and expel any air bubbles. Repeat if air bubbles are present.

### Running the TaqMan Assay

Place the PCR plate in the ABI Prism 7900 and run the cycle. The cycle conditions are as follows: 50° C. for 2 minutes, followed by 95° C. for 10 minutes, 40 cycles at 95° C. for 15 seconds and 53.6° C. for 1 minute. Analyse the data according to manufacturers' instructions and local procedures.

### Analysing Results

Only trained operators should analyse and interpret results. There are a number of software options available for viewing and analysing data.

Internal control pUC18 Ct values indicate whether the reaction has been successful or been inhibited by the presence of polymerase inhibitors. Ct values outside of acceptable range (24.5 cycles to 11.9 cycles) would indicate inhibition and the sample in question should be re-tested. If subsequent tests also generate internal control values outside the acceptable range then the result should be recorded as inconclusive. NTC amplifications should have a Ct value of between 39 and 40. If fluorescence occurs it may be due to thermal degradation of probe and the test should be repeated.

For the general screening assay: Avian DNA has highest threshold cycles when compared to bovine, ovine and porcine DNA, therefore six avian positive control Ct values are used to generate a cut off for this assay. The mean of the 6 well±3 standard deviation (SD) is used to calculate a cut off value. A sample generating a Ct value less than or equal to this value is recorded as positive. A sample with a Ct value greater than this cut off is recorded as negative.

For the species specific assay: The Ct values of 6 wells used for species specific positive control are used to determine the cut off value. The mean of the 6 well±3 standard deviation (SD) is used to calculate a cut off value. A sample generating a Ct value less than or equal to this value is recorded as positive. A sample with a Ct value greater than this cut off is recorded as negative.

Although the present invention has been described with reference to preferred or exemplary embodiments, those skilled in the art will recognize that various modifications and variations to the same can be accomplished without departing from the spirit and scope of the present invention and that such modifications are clearly contemplated herein. No limitation with respect to the specific embodiments disclosed herein and set forth in the appended claims is intended nor should any be inferred.

All documents cited herein are incorporated by reference in their entirety, whether or not this has been specifically stated for each reference.

1. A method for extracting nucleic acid from a sample, comprising the steps of:
(i) incubating between about 30 to 250 g, for example about 35 to 45 g, or about 40 g, of the sample in an incubation buffer;
(ii) autoclaving the sample incubated in step (i); and
(iii) mixing the sample or a portion of the sample autoclaved in step (ii) with a metal-chelating agent, for example a metal-chelating ion-exchange resin.

2. The method according to claim 1, further comprising the step of:
(iv) sedimenting the metal-chelating agent from the sample or portion in step
(iii) to produce a supernatant comprising extracted nucleic acid.

3. The method according to either of claim 1, in which the incubation buffer is a phosphate buffer, for example a sodium phosphate buffer at a concentration of about 0.05 to 0.2 M or about 0.1 M.

4. The method according to claim 1, in which the incubation buffer is about 500 to 1,500% (v/w) of the mass of the sample, for example about 900% (v/w) of the mass of the sample.

5. The method according to claim 1, in which the incubation buffer, for example a sodium phosphate buffer, has a pH of between about 6.5 and 7.5, or about 7.2.

6. The method according to claim 1, in which incubation in step (i) is conducted for about 5 to 60 minutes or for about 10 to 30 minutes, for example for about 15 minutes.

7. The method according to claim 1, in which the sample is autoclaved in step (ii) at about 85 to 120° C. or at about 90 to 100° C., for example at about 95° C., and optionally for about 5 to 60 minutes or for about 10 to 30 minutes, for example for about 15 minutes.

8. The method according to claim 1, in which the sample is autoclaved in step (ii) at or near atmospheric pressure.

9. The method according to claim 1, in which the metal-chelating s agent chelates polyvalent metal ions, for example a resin which is a styrene divinylbenzene copolymer containing paired iminodiacetate ions, such as Chelex 100.

10. The method according to claim 2, in which the metal-chelating agent, for example a metal-chelating ion-exchange resin, is sedimented in step (iv) by centrifugation, for example at about 10,000 to 12,000×g, or about 11,000×g.

11. The method according to claim 1, in which the sample is from animal feed, for example ruminant feed.

12. The method according to claim 1, in which the sample contains or is to be tested for the presence of rendered animal material, for example processed animal proteins (PAPs) or meat and bone meal (MBM).

13. The method according to claim 1, in which the sample comprises animal or ruminant mitochondrial DNA, for example mitochondrial DNA encoding a 16S RNA gene.

14. The method according to claim 1, in which the sample comprises DNA from one or more of the group consisting of: animal, ruminant, bovine, ovine, porcine, avian and piscine tissue.

15. The method according to claim 1, in which the sample is animal feed, and the method comprises the steps of:
(i) incubating between about 35 to 45 g of the sample in a 0.1 M sodium phosphate buffer at a pH of about 7.1 to 7.3 for about 10 to 20 minutes, for example about 15 minutes, optionally at ambient temperature;
(ii) autoclaving the sample incubated in step (i) at or near atmospheric pressure at about 90 to 100° C. for about 10 to 30 minutes, for example for about 15 minutes; and
(iii) mixing a portion of the sample autoclaved in step (ii) with a metal-S chelating ion-exchange resin, for example Chelex 100.

16. The method according to claim 15, comprising the further step of:
(iv) sedimenting the resin from the portion of the sample in step (iii) by centrifugation at about 10,000 to 12,000×g, or at about 11,000×g.

17. The method according to claim 2, in which the supernatant or an aliquot thereof from step (iv) is used in an amplification reaction such as a polymerase chain reaction (PCR) or real-time PCR assay.

18. The method according to claim 1, further comprising the step of:
(v) detecting the presence or absence of nucleic acid, for example DNA such as mitochondrial DNA.

19. The method according to claim 18, in which the nucleic acid is from rendered animal material, for example PAPs or MBM.

20. The method according to claim 18, in which the nucleic acid is detected in step (v) using PCR or real-time PCR.

21. A method for amplifying DNA in a sample, comprising the steps of:
(i) extracting DNA from the sample according to the method defined in claim 1; and
(ii) amplifying the extracted DNA using an amplification reaction such as PCR or real-time PCR.

22. The method according to claim 21, in which the DNA amplified in step (ii) is from rendered animal material, for example PAPs or MBM.

23. The method according to claim 21, in which the PCR or real-time PCR employs a pair of primers, and in the case of real-time PCR additionally employs a probe, in which the primers and probe are specific for one or more of the following: animal, ruminant, bovine, ovine, porcine, avian and piscine tissue.

24. The method according to claim 23, in which the pair of primers and probe are specific for mitochondrial DNA, for example mitochondrial DNA encoding a 16S RNA gene, from one or more of the group consisting of: animal, ruminant, bovine, ovine, porcine, avian and piscine tissue.

25. The method according to claim 24, in which the pair of primers and probe, respectively, are selected from the group of sequences consisting of SEQ ID) NOs 1, 2 and 3 (animal-specific), SEQ ID NOs 4, 5 and 6 (bovine-specific), SEQ ID NOs 7, 8 and 9 (ovine-specific), SEQ ID) NOs 10, 11 and 12 (porcinespecific), SEQ ID) NOs 13, 14 and 15 (avian-specific), and SEQ ID NOs 16, 17 and 18 (piscine-specific).

26. The method according to claim 24, in which the pair of primers and probe are specific for mitochondrial DNA encoding a 16S RNA gene from one or more of the group consisting of: porcine, avian and piscine tissue.

27. The method according to claim 26, in which the pair of primers and probe, respectively, are selected from the group of sequences consisting of SEQ ID NOs 10, 11 and 12 (porcine-specific), SEQ ID NOs 13, 14 and 15 (avianspecific), and SEQ ID) NOs 16, 17 and 18 (piscine-specific).

28. The method according to claim 20, which the PCR or real-time PCR further employs a set of primers, and in the ease of real-time PCR additionally employs a probe, wherein the set of primers and probe are specific to DNA exogenous to the sample, for example the sequences as set forth in SEQ ID NOs 19, 20 and 21.

29. The method according to claim 17, in which the real-time PCR is performed using a TaqMan probes.

30. The method according to claim 1, which excludes the use of magnetic DNA purification, for example the Wizard Magnetic DNA Purification System for Food obtained from Promega (Madison, Wis., USA).

31. The method according to claim 1, which excludes the use of RNAse.

32. The method according to claim 1, which excludes the use of Whatman FTA™ card.

33. The method according to claim 1, in which the incubation buffer excludes a detergent such as Triton X-100.

34. A kit for amplifying DNA from a sample using PCR, comprising a pair of primers selected from the group of sequences consisting of: SEQ ID) NOs 1 and 2 (animal-specific), SEQ ID NOs 4 and 5 (bovine-specific), SEQ ID NOs 7 and 8 (ovine-specific), SEQ ID NOs 10 and 11 (porcine-specific), SEQ ID NOs 13 and 14 (avian-specific), and SEQ ID NOs 16 and 17 (piscine-specific), and optionally further comprising a set of primers specific for DNA exogenous to the sample, for example the sequences as set forth in SEQ ID) NOs 19 and 20.

35. A kit for amplifying DNA from a sample using real-time PCR, comprising a one pair of primers and a probe, respectively, selected from the group of sequences consisting of: SEQ U) NOs 1, 2 and 3 (animal-specific), SEQ 11) NOs 4, 5 and 6 (bovine-specific), SEQ U) NOs 7, 8 and 9 (ovine-specific), SEQ ID NOs 10, 11 and 12 (porcine-specific), SEQ II) NOs 13, 14 and 15 (avian-specific), and SEQ ID NOs 16, 17 and 18 (piscine-specific), and optionally further comprising a set of primers and a probe specific to DNA exogenous to the sample, for example the sequences as set forth in SEQ ID NOs 19, 20 and 21.

36. The kit according to claim 34, for amplifying rendered animal material, for example PAPs or MBM, from a feed sample.

37. An isolated nucleic acid comprising one or more of the sequences set forth in SEQ ID) NOs 1-21, for example the sequences set forth in SEQ ID NOs 10-18.

38. Extracted nucleic acid obtainable according to the method of claim 1.

39. A method for extracting nucleic acid from a sample, substantially as described herein.

40. A method for amplifying DNA in a sample, substantially as described herein.

41. A kit for amplifying DNA from a sample, substantially as described herein.
